(19) United States
(12) Patent Application Publication
(10) Pub. No.: US 2026/0151547 A1
(43) Pub. Date: Jun. 4, 2026

(54) FILTER DEVICE

(71) Applicant: TERUMO KABUSHIKI KAISHA, Tokyo (JP)

(72) Inventor: Naoki IIDA, Fujinomiya-shi Shizuoka (JP)

(21) Appl. No.: 19/420,285

(22) Filed: Dec. 15, 2025

Related U.S. Application Data
(63) Continuation of application No. PCT/JP2024/021796, filed on Jun. 17, 2024.

(30) Foreign Application Priority Data
Jun. 16, 2023 (JP) 2023099354

(51) Int. Cl. A61M 1/02; B04B 3/00; B04B 5/04

(52) U.S. Cl. CPC A61M 1/0281; A61M 1/0272; B04B 3/00; B04B 5/0442; A61M 2202/0439; A61M 2205/125; B04B 2005/0478

(57) ABSTRACT

A filter device according to the present invention is to be mounted on a centrifugal separator and includes: a first space which is formed in a housing and into which blood flows; a second space which is formed in the housing and from which blood flows out; a partition wall that separates the first space and the second space and that is formed with a communication port establishing communication between the first space and the second space; and a white-blood-cell removal filter that is disposed in the second space. The first space is located between the partition wall and a first main surface of the housing, and the second space is located between the partition wall and a second main surface of the housing.

### CROSS-REFERENCE TO RELATED APPLICATIONS

The present disclosure is a continuation of and claims benefit to PCT/JP2024/021796 filed on Jun. 17, 2024, entitled “FILTER DEVICE” which claims priority to Japanese Patent Application No. 2023-099354, filed on Jun. 16, 2023. The entire disclosure of the applications listed above are hereby incorporated herein by reference, in their entireties, for all that they teach and for all purposes.

### BACKGROUND

The present disclosure relates to a filter device.

Japanese Patent No. 5223006 B2 describes a blood bag system and a centrifugal transfer device (e.g., centrifugal separator). The blood bag system accommodates blood. The centrifugal separator centrifuges the blood accommodated in the blood bag system. The centrifuged blood is used for blood transfusion.

### BRIEF SUMMARY

It is preferable that blood to be transfused does not contain white blood cells. Recently, a filter device capable of more suitably removing white blood cells is desired.

It is an object of the present disclosure to solve the problem described above.

One aspect of the present disclosure is (1) a filter device to be mounted on a centrifugal separator, the filter device including: a first space which is formed in a housing and into which blood flows through an inlet port, wherein the first space comprises a curved wall that faces the inlet port; a second space which is formed in the housing and from which the blood flows out through an outlet port; a partition wall that separates the first space and the second space and that is formed with a communication port establishing communication between the first space and the second space; and a white-blood-cell removal filter that is disposed in the second space and that removes a white blood cell contained in the blood, wherein the first space is located between a first main surface of the housing and the partition wall formed along the first main surface, wherein the second space is located between a second main surface of the housing and the partition wall, the second main surface extending along the first main surface, and wherein a blood flow path is defined extending from the inlet port into the filter device in a first direction toward the curved wall in the first space, through the communication port in a second direction, through the white-blood-cell removal filter in the second space, and through the second space in a third direction toward the outlet port.

With this configuration, white blood cells can be suitably removed from blood.

Aspect (2) describes the filter device according to aspect (1), in which the inlet port may be located between a center line of the housing along a first direction and a rotation center of the centrifugal separator, the first direction intersecting a centrifugal direction in which a centrifugal force is applied.

With this configuration, the blood can smoothly flow into the housing by the centrifugal force.

Aspect (3) describes the filter device according to aspect (1) or aspect (2), wherein the curved wall is located in a centrifugal direction in which a centrifugal force is applied relative to the inlet port and the communication port, and wherein the third direction is opposite the first direction.

This configuration makes it possible to prevent backflow of blood toward the inlet port.

Aspect (4) describes the filter device according to any one of aspects (1) to (3) provided above, in which the first space may be provided with a meandering flow channel for allowing the blood flowing in through the inlet port to meander.

This configuration makes it possible to prevent blood from which white blood cells have not been removed by centrifugation from reaching the second space.

Aspect (5) describes the filter device according to any one of aspects (1) to (4) provided above, in which the communication port may be located on one side of the second space in a centrifugal direction in which a centrifugal force is applied, and the outlet port may be located on another side of the second space in the centrifugal direction.

With this configuration, a sufficient distance between the communication port and the outlet port in the centrifugal direction can be obtained.

Aspect (6) describes the filter device according to aspect (5), in which a direction from the outlet port toward the communication port is at least one of a direction along the centrifugal direction and a direction opposite the centrifugal direction.

This configuration makes it possible to prevent white blood cells from reaching the outlet port.

Aspect (7) describes the filter device according to aspect (1), further comprising an inhibition wall disposed in the first space and positioned between the communication port and the inlet port, wherein the inhibition wall comprises a first portion that extends along the first direction and a second portion that extends along a direction perpendicular to the first direction, and wherein the blood flow path extends around the inhibition wall prior to extending through the communication port in the second direction.

With this configuration, the blood can be suitably guided toward the outlet port by the centrifugal force.

Aspect (8) describes the filter device according to any one of aspects (1) to (7) provided above, in which a thickness direction may be along a vertical direction in a state where the filter device is mounted on the centrifugal separator.

With this configuration, the dimension of the filter device in the horizontal direction can be shortened as compared with the case where the first space and the second space are adjacent to each other in the horizontal direction.

One aspect of the present disclosure is (9) a filter device, comprising: a housing comprising a width extending along a first direction from a first width side of the filter device to a second width side of the filter device, a thickness extending along a second direction from a first main surface of the housing to a second main surface of the housing, and a length extending along a centrifugal direction from a first end of the filter device to a second end of the filter device, wherein a center of the length defines a center line extending along the first direction bisecting the length between the first end of the filter device and the second end of the filter device, and wherein the housing further comprises: a first space disposed inside the housing adjacent the first main surface of the housing, wherein the first space comprises a curved interior wall arranged adjacent the first width side of the filter device and the second end of the filter device; a second space disposed inside the housing adjacent the second main surface of the housing; a partition wall arranged between the first space and the second space, wherein the partition wall separates the first space from the second space; a communication port arranged in the partition wall, wherein the communication port fluidly interconnects the first space and the second space; an inlet port arranged in fluid communication with the first space; and an outlet port arranged in fluid communication with the second space; wherein, when a centrifugal force is applied to the filter device in the centrifugal direction, a blood flow path is defined extending from the inlet port into the filter device along the centrifugal direction toward the curved interior wall in the first space, through the communication port along the second direction, and through the second space and to the outlet port in a centripetal direction opposite the centrifugal direction.

Aspect (10) describes the filter device according to aspect (9), in which the filter device further comprises: a white-blood-cell removal filter disposed inside the second space, wherein the white-blood-cell removal filter comprises a filter medium that is configured to remove white blood cells from a portion of blood flowing through the white-blood-cell removal filter. Aspect (11) describes the filter device according to aspect (9) or aspect (10), in which the second space is arranged closer to the first end of the filter device than the second end of the filter device. Aspect (12) describes the filter device according to any one of aspects (9) to (11), in which a portion of the first space at least partially overlaps a portion of the second space measured along the length and the width of the housing. Aspect (13) describes the filter device according to any one of aspects (9) to (12) further comprising an inflow channel extending inside the housing between the inlet port and the first space, wherein the inflow channel extends along the centrifugal direction; and an inhibition wall disposed in the first space and positioned between the communication port and the inlet port, wherein the inhibition wall extends along the first direction, and wherein the blood flow path extends around the inhibition wall prior to extending through the communication port. Aspect (14) describes the filter device according to any one of aspects (9) to (13), in which the inlet port extends between an exterior of the housing and an interior of the housing at the first end of the filter device. Aspect (15) describes the filter device according to any one of aspects (9) to (14), in which the outlet port extends between the exterior of the housing and the interior of the housing at the first end of the filter device. Aspect (16) describes the filter device according to any one of aspects (9) to (15), in which a center of the thickness defines a vertical center of the housing, and wherein the partition wall is arranged at the vertical center of the housing spanning along the first direction and the centrifugal direction. Aspect (17) describes the filter device according to any one of aspects (9) to (16), in which the vertical center of the housing divides the housing into a lower vertical portion of the housing and an upper vertical portion of the housing measured along the second direction, and wherein the first space is arranged in the lower vertical portion of the housing, and wherein the second space is arranged in the upper vertical portion of the housing. Aspect (18) describes the filter device according to any one of aspects (9) to (17), in which an inflow direction defined by the inlet port is arranged extending in the centrifugal direction, wherein an outflow direction defined by the outlet port is arranged extending in the centripetal direction, and wherein the inflow direction and the outflow direction are arranged parallel to one another.

One aspect of the present disclosure is (19) a filter device, comprising: a housing comprising a width extending along a first direction from a first width side of the filter device to a second width side of the filter device, a thickness extending along a second direction from a first main surface of the housing to a second main surface of the housing, and a length extending along a centrifugal direction from a first end of the filter device to a second end of the filter device, wherein a center of the length defines a center line extending along the first direction bisecting the length between the first end of the filter device and the second end of the filter device, and wherein the housing further comprises: a first space disposed inside the housing adjacent the first main surface of the housing, wherein the first space comprises a curved interior wall arranged adjacent the first width side of the filter device and the second end of the filter device; a second space disposed inside the housing adjacent the second main surface of the housing; a partition wall arranged between the first space and the second space, wherein the partition wall separates the first space from the second space; a communication port arranged in the partition wall, wherein the communication port fluidly interconnects the first space and the second space; an inlet port arranged in fluid communication with the first space; an outlet port arranged in fluid communication with the second space; and a white-blood-cell removal filter disposed inside the second space, wherein the white-blood-cell removal filter comprises a filter medium that is configured to remove white blood cells from a portion of blood flowing through the white-blood-cell removal filter; wherein a fluid flow path is defined extending from the inlet port to the first space, from the first space through the communication port to the second space, and through the second space to the outlet port.

Aspect (20) describes the filter device according to aspect (19), further comprising an inhibition wall disposed in the first space and positioned between the communication port and the inlet port, wherein the inhibition wall extends along the first direction, and wherein the fluid flow path extends around the inhibition wall prior to extending from the first space through the communication port to the second space.

According to the present disclosure, white blood cells can be suitably removed from blood.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a perspective view illustrating a centrifugal separator to which a filter device according to embodiments of the present disclosure is mounted.

FIG. 2 is a perspective view illustrating the filter device in accordance with embodiments of the present disclosure.

FIG. 3 is a cross-sectional view of the filter device in accordance with embodiments of the present disclosure.

FIG. 4 is a plan view illustrating a first space formed in a housing in accordance with embodiments of the present disclosure.

FIG. 5 is a plan view illustrating a second space formed in the housing in accordance with embodiments of the present disclosure.

FIG. 6 is a plan view illustrating a first space formed in a housing according to a first modification of the present disclosure.

FIG. 7 is a plan view illustrating a second space formed in the housing according to the first modification of the present disclosure.

FIG. 8 is a plan view illustrating a first space formed in a housing according to a second modification of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

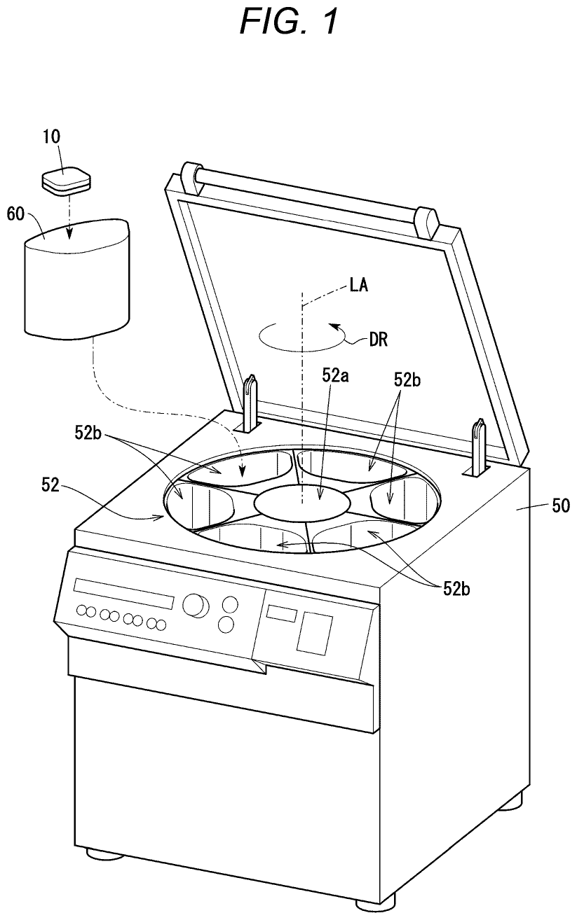

A filter device according to at least one embodiment of the present disclosure will be described with reference to the drawings. FIG. 1 is a perspective view illustrating a centrifugal separator 50 to which a filter device 10 according to a first embodiment is to be mounted.

The centrifugal separator 50 is a machine that centrifugally separates blood. The centrifugal separator 50 includes a centrifugal drum 52. The centrifugal drum 52 includes a central body 52a and a plurality of unit insertion portions 52b (e.g., receivers).

The plurality of unit insertion portions 52b are disposed so as to surround the central body 52a. An insert unit 60 (e.g., container, receptacle, etc.) can be inserted into each of the plurality of unit insertion portions 52b. The insert unit 60 is attached to the centrifugal separator 50 by being inserted into the corresponding unit insertion portion 52b.

A blood bag system (not illustrated) may be accommodated in the insert unit 60. The blood bag system may include a blood bag containing blood before being centrifuged. The blood contained in the blood bag is, for example, whole blood, but may be buffy coat as described in Japanese Patent No. 5223006 B2 above. The buffy coat contains red blood cells, platelets, white blood cells, and the like.

The centrifugal separator 50 rotates the insert unit 60 inserted into the unit insertion portion 52b about the central body 52a. More specifically, the centrifugal separator 50 rotates the insert unit 60 inserted into the unit insertion portion 52b along a rotation direction DR about the center line of rotation (e.g., rotation center) LA illustrated in FIG. 1. Accordingly, a centrifugal force is applied to the entire insert unit 60. The blood is centrifuged by the centrifugal force. The center line of rotation LA is, for example, along a gravity direction (e.g., second direction D2 to be described later).

The filter device 10 is mounted on the insert unit 60. The filter device 10 is mounted to, for example, but not limited to, an upper part of the insert unit 60.

The filter device 10 is mounted to the insert unit 60, and thus is mounted to the centrifugal separator 50 via the insert unit 60. The centrifugal force described above is applied not only to the insert unit 60 but also to the filter device 10. Note that the filter device 10 may be directly mounted to the centrifugal separator 50.

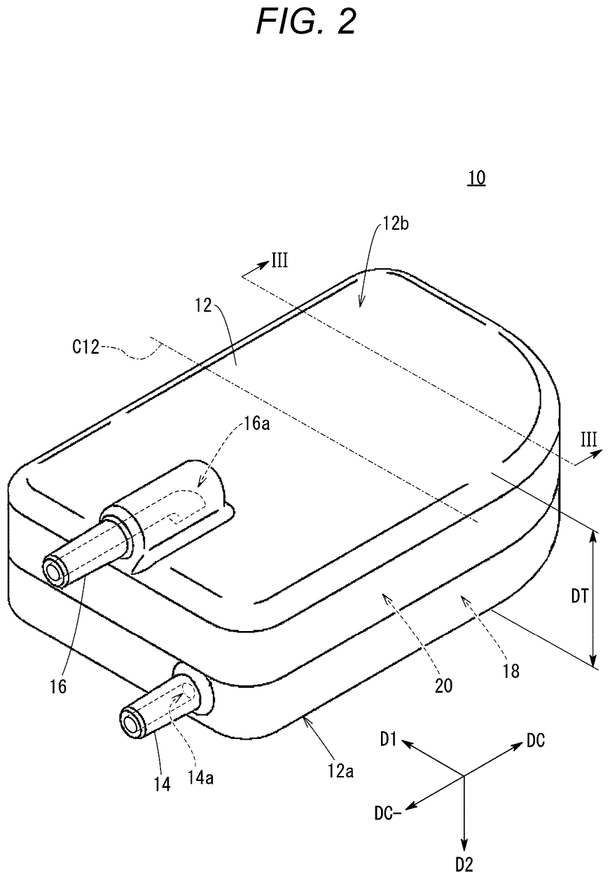

FIG. 2 is a perspective view illustrating the filter device 10 in accordance with embodiments of the present disclosure.

As described above, the filter device 10 may be mounted to the centrifugal separator 50. In the following, the configuration of the filter device 10 will be described based on the premise that the filter device 10 is mounted to the centrifugal separator 50.

As illustrated in FIG. 2, reference may be made to a centrifugal direction DC, a centripetal direction DC−, a first direction D1, and a second direction D2. The centrifugal direction DC is a direction of the centrifugal force applied to the filter device 10 by the centrifugal separator 50. The first direction D1 is a direction of a tangential velocity in the circular movement of the filter device 10 by the centrifugal separator 50. The first direction D1 is orthogonal to (e.g., intersects) the centrifugal direction DC. In some embodiments, the first direction D1 may be arranged perpendicular to the centrifugal direction DC. The second direction D2 is a gravity direction (e.g., shown as the vertical direction in FIG. 2). In the present embodiment, the centrifugal direction DC and the first direction D1 are orthogonal to the second direction D2. The centripetal direction DC− is a direction opposite to the centrifugal direction DC.

The filter device 10 includes a housing 12. The housing 12 can be formed in, for example, a box shape. In the present embodiment, in a state where the filter device 10 is mounted to the centrifugal separator 50, the thickness direction DT of the housing 12 coincides with (e.g., is arranged parallel to) the second direction D2.

The housing 12 has a first main surface 12a and a second main surface 12b. The first main surface 12a is an outer surface on one side of the housing 12 in the thickness direction DT. The second main surface 12b is an outer surface on the other side of the housing 12 in the thickness direction DT. The first main surface 12a and the second main surface 12b extend along the centrifugal direction DC and the first direction D1.

In the present embodiment, the first main surface 12a corresponds to a lower surface of the housing 12. In the present embodiment, the second main surface 12b corresponds to an upper surface of the housing 12.

The housing 12 includes an inlet portion 14. The inlet portion 14 includes an inlet port 14a. The inlet port 14a may correspond to an opening that establishes communication (e.g., fluid communication, etc.) between the inside and the outside of the housing 12. The inlet port 14a is located in the centripetal direction DC− relative to a center line C12 of the housing 12 along the first direction D1. In other words, the inlet port 14a is positioned between the center line C12 and the rotation center LA (see, e.g., FIG. 1) of the centrifugal separator 50. The inlet portion 14 illustrated in FIG. 2 protrudes from the housing 12, but is not limited thereto.

The housing 12 further includes an outlet portion 16. The outlet portion 16 has an outlet port 16a. The outlet port 16a is an opening that establishes communication (e.g., fluid communication, etc.) between the inside and the outside of the housing 12. The outlet portion 16 illustrated in FIG. 2 protrudes from the housing 12, but is not limited thereto.

Different blood bags may be connected to the inlet portion 14 and the outlet portion 16. The blood bag connected to the inlet portion 14 is a blood bag containing, in advance, blood before being centrifuged. The blood bag connected to the outlet portion 16 is a blood bag for accommodating blood (e.g., a blood component) that has passed through the filter device 10.

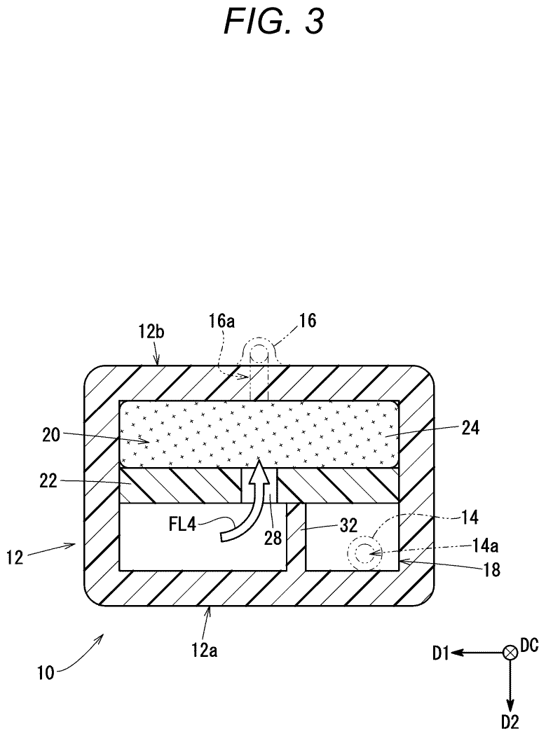

FIG. 3 is a cross-sectional view of the filter device 10 in accordance with embodiments of the present disclosure. FIG. 3 illustrates a cross-sectional view taken along line III-III in FIG. 2.

The housing 12 includes a first space 18 (e.g., first interior space), a second space 20 (e.g., second interior space), and a partition wall 22.

The first space 18 is a space located between the first main surface 12a and the partition wall 22. The first space 18 is in communication with the inlet port 14a. On the other hand, the second space 20 is a space located between the partition wall 22 and the second main surface 12b. The second space 20 is in communication with the outlet port 16a.

The partition wall 22 extends along the centrifugal direction DC and the first direction D1 similarly to the first main surface 12a, the second main surface 12b, and the like. The first space 18 and the second space 20 are separated by the partition wall 22. The partition wall 22 can define an upper surface of the first space 18 and a bottom surface of the second space 20.

The first space 18 is provided with an inhibition wall 32. The partition wall 22 is formed with a communication port 28. The communication port 28 and the inhibition wall 32 will be described later.

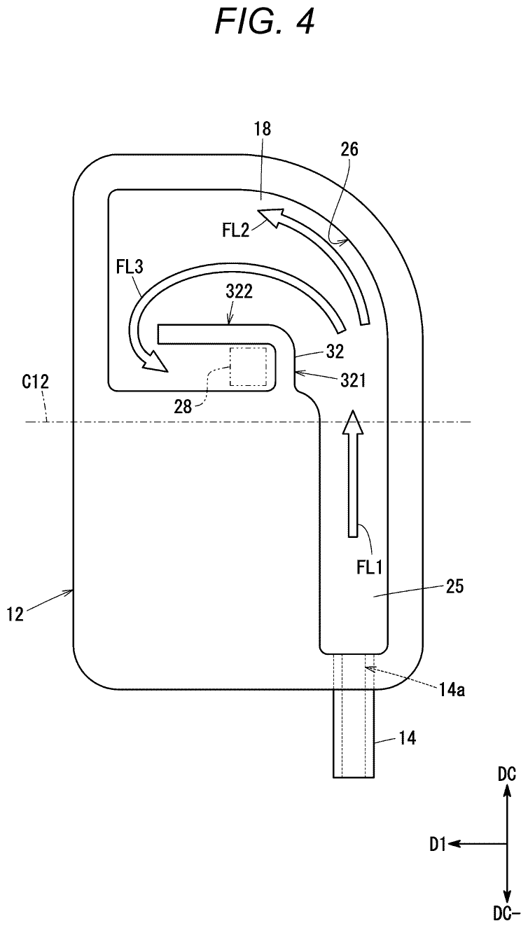

FIG. 4 is a plan view illustrating the first space 18 formed in the housing 12 in accordance with embodiments of the present disclosure.

As described above, the first space 18 is arranged in communication (e.g., fluid communication, etc.) with the inlet port 14a. For example, blood flows into the first space 18 through the inlet port 14a.

The inlet port 14a is preferably formed along the centrifugal direction DC. In this case, the blood smoothly flows through the inlet port 14a and flows into the first space 18 by the centrifugal force applied by the centrifugal separator 50.

An inflow channel 25 can be formed in the first space 18. The inflow channel 25 is a flow path extending from the inlet port 14a along the centrifugal direction DC. The blood flowing into the first space 18 through the inlet port 14a can flow in the centrifugal direction DC along the inflow channel 25 (FL1).

The first space 18 may include a curved wall 26 (e.g., curved interior wall). The curved wall 26 is positioned in the centrifugal direction DC relative to the inlet port 14a and the communication port 28 described above. The curved wall 26 is curved so as to guide blood from one side to the other side of the first space 18 in the first direction D1. The blood flows along the curved wall 26. Therefore, the blood can flow from one side to the other side of the first space 18 in the first direction D1 without flowing back toward the inlet port 14a (FL2, FL3)

The blood flowing into the first space 18 is centrifuged by the centrifugal separator 50. As a result, the blood in the first space 18 has a layer of a supernatant liquid and a layer of a sedimentation liquid. The layer of the sedimentation liquid is located in the centrifugal direction DC relative to the layer of the supernatant liquid.

The main component of the sedimentation liquid is white blood cells. The reason why white blood cells are considered to be the main component of the sedimentation liquid is that white blood cells are relatively heavy blood components. That is, white blood cells are blood components that are relatively easily precipitated, and thus, are main components of the sedimentation liquid. On the other hand, the supernatant liquid contains a blood component lighter than white blood cells. For example, the supernatant liquid contains large quantities of platelets and the like.

The communication port 28 is an opening that establishes communication (e.g., fluid communication, etc.) between the first space 18 and the second space 20. As described above, the communication port 28 is formed in the partition wall 22. The blood can flow from the first space 18 into the second space 20 through the communication port 28 (see, e.g., FL4 in FIG. 3).

The inhibition wall 32 inhibits the blood flowing into the first space 18 through the inlet port 14a from immediately reaching the communication port 28. In order for the blood to reach the communication port 28 from the inlet port 14a, the blood may need to bypass the inhibition wall 32. The inhibition wall 32 includes a first inhibition wall portion 321 and a second inhibition wall portion 322. The first inhibition wall portion 321 is a wall portion extending in the centrifugal direction DC. The first inhibition wall portion 321 is positioned between the communication port 28 and the inlet port 14a in the first direction D1. The second inhibition wall portion 322 is a wall portion extending in the first direction D1 from the first inhibition wall portion 321.

The second inhibition wall portion 322 illustrated in FIG. 4 protrudes in the first direction D1 from the leading end of the first inhibition wall portion 321, but is not limited thereto. The second inhibition wall portion 322 may not be provided.

Here, as described above, the white blood cells are collected on the side in the centrifugal direction DC in the first space 18 by the centrifugation with the centrifugal separator 50 (FL2). In other words, the white blood cells are less likely to flow in the centripetal direction DC− during the centrifugation with the centrifugal separator 50. Therefore, the white blood cells, once flowing in the centrifugal direction DC from the communication port 28, are less likely to reach the communication port 28.

On the other hand, as described above, blood components other than the white blood cells can flow farther to the side in the centripetal direction DC− than the white blood cells due to the centrifugation with the centrifugal separator 50. Therefore, the blood components other than the white blood cells can reach the communication port 28 even after flowing in the centrifugal direction DC through the communication port 28 (FL3).

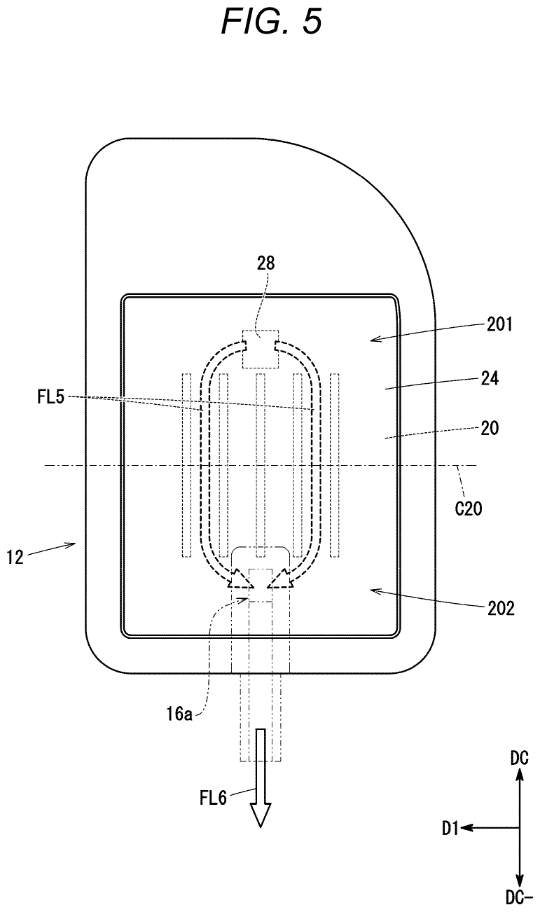

FIG. 5 is a plan view illustrating the second space 20 formed in the housing 12 in accordance with embodiments of the present disclosure.

A white-blood-cell removal filter 24 is provided in the second space 20 (see also FIG. 3). The white-blood-cell removal filter 24 includes a filter medium for removing white blood cells from blood. The blood that flows into the second space 20 is filtered by the white-blood-cell removal filter 24 (see, e.g., FL5 in FIG. 5). Thus, even if white blood cells flow into the second space 20, the white blood cells can be removed by the white-blood-cell removal filter 24.

As described above, the second space 20 is in communication with the outlet port 16a. Therefore, the blood in the second space 20 can flow out of the housing 12 through the outlet port 16a (FL6).

The second space 20 includes a first section 201 and a second section 202. The first section 201 is a region on one side of the second space 20 in the centrifugal direction DC. On the other hand, the second section 202 is a region on the other side of the second space 20 in the centrifugal direction DC. More specifically, the first section 201 is a region on the side in the centrifugal direction DC relative to the center line C20 in the second space 20. On the other hand, the second section 202 is a region on the side in the centripetal direction DC− relative to the center line C20 in the second space 20. The center line C20 is a virtual straight line that passes through the center of the second space 20 and is parallel to the first direction D1.

The communication port 28 is located in the first section 201. On the other hand, a communicating portion between the outlet port 16a and the second space 20 is located in the second section 202. With this configuration, a flow path of blood sufficient for removing white blood cells by the white-blood-cell removal filter 24 can be provided between the communication port 28 and the outlet port 16a. As a result, white blood cells contained in the blood can be more reliably removed by the white-blood-cell removal filter 24 before the blood reaches the outlet port 16a from the communication port 28.

In order for the blood flowing into the second space 20 to reach the outlet port 16a, the blood needs to move in the centripetal direction DC− from the communication port 28. As described above, the white blood cells are less likely to flow in the centripetal direction DC− during the centrifugation with the centrifugal separator 50. That is, since the direction from the outlet port 16a toward the communication port 28 is the centrifugal direction DC as illustrated in FIG. 5, it is possible to prevent white blood cells from reaching the outlet port 16a. Thus, the amount of white blood cells contained in the blood taken out from the outlet port 16a can be more suitably reduced.

As described above, according to the present embodiment, the blood flowing into the first space 18 is centrifuged by the centrifugal separator 50. Thus, the amount of white blood cells contained in the blood flowing into the second space 20 is reduced. The white blood cells remaining in the blood flowing into the second space 20 are removed by the white-blood-cell removal filter 24. That is, according to the present embodiment, the removal of white blood cells by centrifugation and the removal of white blood cells by the white-blood-cell removal filter 24 can be achieved on blood only by operating the centrifugal separator 50.

Modifications according to the above embodiment will be described below. Note that the description overlapping with the above embodiment will be appropriately omitted. Elements described in the above embodiment are denoted by the same reference numerals as those in the above embodiment unless otherwise specified.

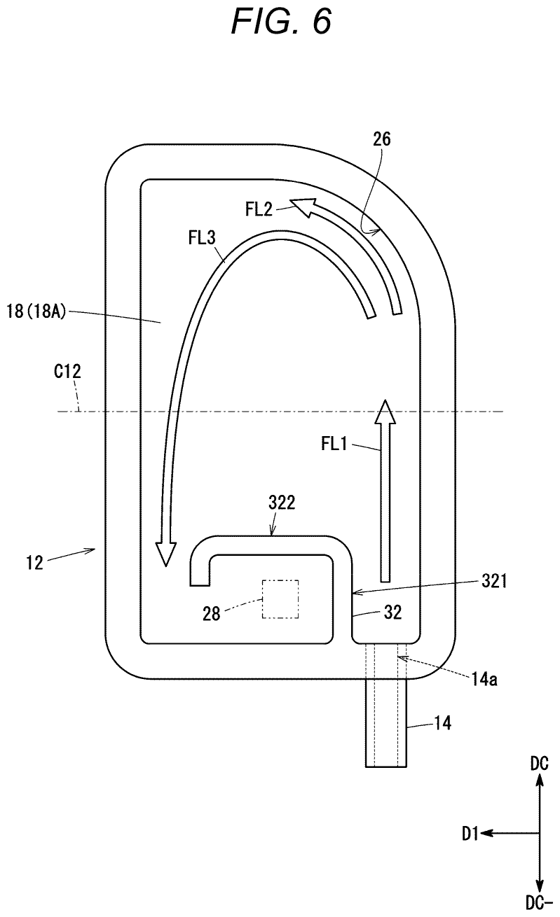

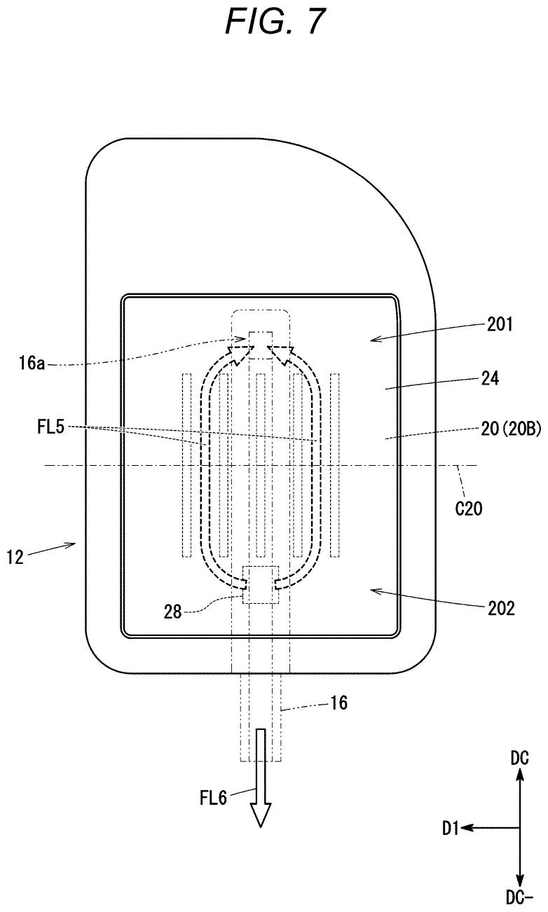

FIG. 6 is a plan view illustrating a first space 18 (18A) formed in a housing 12 according to the first modification. FIG. 7 is a plan view illustrating a second space 20 (20B) formed in the housing 12 according to the first modification.

In this first modification, the communication port 28 is located on the side in the centrifugal direction DC relative to the center line C12 of the housing 12, but the present invention is not limited thereto. As illustrated in FIG. 6, the communication port 28 may be located on the side in the centripetal direction DC− relative to the center line C12. The communication port 28 is located as far as possible in the centripetal direction DC− in the first space 18A, by which it is possible to prevent white blood cells from reaching the communication port 28.

As illustrated in FIG. 7, the communication port 28 may be located in a second section 202 of the second space 20B. In a case where the communication port 28 is located in the second section 202 of the second space 20B, a communicating portion between the outlet port 16a and the second space 20B may be located in a first section 201. With this configuration, a flow path of blood sufficient for removing white blood cells by the white-blood-cell removal filter 24 can also be provided between the communication port 28 and the outlet port 16a.

In the present modification, the direction from the communication port 28 toward the outlet port 16a is along the centrifugal direction DC (see, e.g., FL5 in FIG. 7). Therefore, the blood flowing into the second space 20B can smoothly reach the outlet port 16a from the communication port 28 by the centrifugal force.

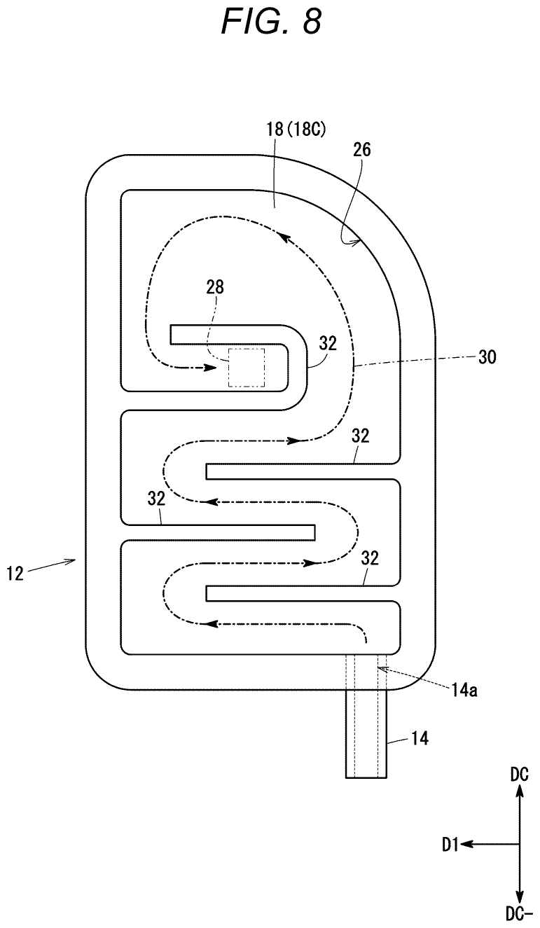

FIG. 8 is a plan view illustrating a first space 18 (18C) formed in a housing 12 according to the second modification.

As illustrated in FIG. 8, a meandering flow channel 30 for allowing blood flowing in through the inlet port 14a to meander is formed in the first space 18C. The meandering flow channel 30 may be constituted by, for example, a plurality of inhibition walls 32.

The blood flowing in through the inlet port 14a reaches the communication port 28 through the meandering flow channel 30. Thus, it is possible to prevent the blood from immediately flowing into the communication port 28 from the inlet port 14a. That is, it is possible to prevent blood from which white blood cells have not been removed by centrifugation from reaching the second space 20. The specific shape of the meandering flow channel 30 is not limited to the example in FIG. 8.

The plurality of modifications described above may be appropriately combined as long as there are no inconsistencies.

Note that the present invention is not limited to the above disclosure and can take various configurations without departing from the gist of the present invention.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Z₀, the phrase is intended to refer to a single element selected from X, Y, and Z, a combination of elements selected from the same class (e.g., X₁ and X₂) as well as a combination of elements selected from two or more classes (e.g., Y₁ and Z₀).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A filter device to be mounted on a centrifugal separator, the filter device comprising:
a first space which is formed in a housing and into which blood flows through an inlet port, wherein the first space comprises a curved wall that faces the inlet port;
a second space which is formed in the housing and from which the blood flows out through an outlet port;
a partition wall that separates the first space and the second space and that is formed with a communication port establishing communication between the first space and the second space; and
a white-blood-cell removal filter that is disposed in the second space and that removes a white blood cell contained in the blood,
wherein the first space is located between a first main surface of the housing and the partition wall formed along the first main surface,
wherein the second space is located between a second main surface of the housing and the partition wall, the second main surface extending along the first main surface, and
wherein a blood flow path is defined extending from the inlet port into the filter device in a first direction toward the curved wall in the first space, through the communication port in a second direction, through the white-blood-cell removal filter in the second space, and through the second space in a third direction toward the outlet port.

2. The filter device of claim 1, wherein the inlet port is located between a center line of the housing along a first direction and a rotation center of the centrifugal separator, the first direction intersecting a centrifugal direction in which a centrifugal force is applied.

3. The filter device of claim 1, wherein the curved wall is located in a centrifugal direction in which a centrifugal force is applied relative to the inlet port and the communication port, and wherein the third direction is opposite the first direction.

4. The filter device of claim 1, wherein the first space is provided with a meandering flow channel for allowing the blood flowing in through the inlet port to meander.

5. The filter device of claim 1, wherein the communication port is located on one side of the second space in a centrifugal direction in which a centrifugal force is applied, and wherein the outlet port is located on another side of the second space in the centrifugal direction.

6. The filter device of claim 5, wherein a direction from the outlet port toward the communication port is at least one of a direction along the centrifugal direction and a direction opposite the centrifugal direction.

7. The filter device of claim 1, further comprising:
an inhibition wall disposed in the first space and positioned between the communication port and the inlet port, wherein the inhibition wall comprises a first portion that extends along the first direction and a second portion that extends along a direction perpendicular to the first direction, and wherein the blood flow path extends around the inhibition wall prior to extending through the communication port in the second direction.

8. The filter device of claim 1, wherein a thickness direction is along a vertical direction in a state where the filter device is mounted on the centrifugal separator.

9. A filter device, comprising:
a housing comprising a width extending along a first direction from a first width side of the filter device to a second width side of the filter device, a thickness extending along a second direction from a first main surface of the housing to a second main surface of the housing, and a length extending along a centrifugal direction from a first end of the filter device to a second end of the filter device, wherein a center of the length defines a center line extending along the first direction bisecting the length between the first end of the filter device and the second end of the filter device, and wherein the housing further comprises:
a first space disposed inside the housing adjacent the first main surface of the housing, wherein the first space comprises a curved interior wall arranged adjacent the first width side of the filter device and the second end of the filter device;
a second space disposed inside the housing adjacent the second main surface of the housing;
a partition wall arranged between the first space and the second space, wherein the partition wall separates the first space from the second space;
a communication port arranged in the partition wall, wherein the communication port fluidly interconnects the first space and the second space;
an inlet port arranged in fluid communication with the first space; and
an outlet port arranged in fluid communication with the second space;
wherein, when a centrifugal force is applied to the filter device in the centrifugal direction, a blood flow path is defined extending from the inlet port into the filter device along the centrifugal direction toward the curved interior wall in the first space, through the communication port along the second direction, and through the second space and to the outlet port in a centripetal direction opposite the centrifugal direction.

10. The filter device of claim 9, further comprising:
a white-blood-cell removal filter disposed inside the second space, wherein the white-blood-cell removal filter comprises a filter medium that is configured to remove white blood cells from a portion of blood flowing through the white-blood-cell removal filter.

11. The filter device of claim 9, wherein the second space is arranged closer to the first end of the filter device than the second end of the filter device.

12. The filter device of claim 9, wherein a portion of the first space at least partially overlaps a portion of the second space measured along the length and the width of the housing.

13. The filter device of claim 9, further comprising:
an inflow channel extending inside the housing between the inlet port and the first space, wherein the inflow channel extends along the centrifugal direction; and
an inhibition wall disposed in the first space and positioned between the communication port and the inlet port, wherein the inhibition wall extends along the first direction, and wherein the blood flow path extends around the inhibition wall prior to extending through the communication port.

14. The filter device of claim 13, wherein the inlet port extends between an exterior of the housing and an interior of the housing at the first end of the filter device.

15. The filter device of claim 14, wherein the outlet port extends between the exterior of the housing and the interior of the housing at the first end of the filter device.

16. The filter device of claim 15, wherein a center of the thickness defines a vertical center of the housing, and wherein the partition wall is arranged at the vertical center of the housing spanning along the first direction and the centrifugal direction.

17. The filter device of claim 16, wherein the vertical center of the housing divides the housing into a lower vertical portion of the housing and an upper vertical portion of the housing measured along the second direction, and wherein the first space is arranged in the lower vertical portion of the housing, and wherein the second space is arranged in the upper vertical portion of the housing.

18. The filter device of claim 17, wherein an inflow direction defined by the inlet port is arranged extending in the centrifugal direction, wherein an outflow direction defined by the outlet port is arranged extending in the centripetal direction, and wherein the inflow direction and the outflow direction are arranged parallel to one another.

19. A filter device, comprising:
a housing comprising a width extending along a first direction from a first width side of the filter device to a second width side of the filter device, a thickness extending along a second direction from a first main surface of the housing to a second main surface of the housing, and a length extending along a centrifugal direction from a first end of the filter device to a second end of the filter device, wherein a center of the length defines a center line extending along the first direction bisecting the length between the first end of the filter device and the second end of the filter device, and wherein the housing further comprises:
a first space disposed inside the housing adjacent the first main surface of the housing, wherein the first space comprises a curved interior wall arranged adjacent the first width side of the filter device and the second end of the filter device;
a second space disposed inside the housing adjacent the second main surface of the housing;
a partition wall arranged between the first space and the second space, wherein the partition wall separates the first space from the second space;
a communication port arranged in the partition wall, wherein the communication port fluidly interconnects the first space and the second space;
an inlet port arranged in fluid communication with the first space;
an outlet port arranged in fluid communication with the second space; and
a white-blood-cell removal filter disposed inside the second space, wherein the white-blood-cell removal filter comprises a filter medium that is configured to remove white blood cells from a portion of blood flowing through the white-blood-cell removal filter;
wherein a fluid flow path is defined extending from the inlet port to the first space, from the first space through the communication port to the second space, and through the second space to the outlet port.

20. The filter device of claim 19, further comprising:
an inhibition wall disposed in the first space and positioned between the communication port and the inlet port, wherein the inhibition wall extends along the first direction, and wherein the fluid flow path extends around the inhibition wall prior to extending from the first space through the communication port to the second space.
